(19) United States
(12) Patent Application Publication
(10) Pub. No.: US 2012/0053653 A1
(43) Pub. Date: Mar. 1, 2012

(54) GASTROINTESTINAL DEVICE

(75) Inventors: Martin Hiernaux, Bruxelles (BE); Nicolas Cauche, Bruxelles (BE); Jacques Deviere, Charleroi (BE); Hubert Louis, Bruxelles (BE)

(73) Assignee: Universite Libre de Bruxelles, Bruxelles (BE)

(21) Appl. No.: 13/319,506

(22) PCT Filed: May 10, 2010
(86) PCT No.: PCT/EP2010/056378

§ 371 Date: Nov. 8, 2011

(30) Foreign Application Priority Data
May 8, 2009 (EP) 091598219

(51) Int. Cl. A61N 1/00

(52) U.S. Cl. 607/40

(57) ABSTRACT

A gastrointestinal device to be implanted into an animal or human tissue includes an elongated body (1) having a first end and a second end, and at least a first abutting member (2) at the first end of the elongated body (1). The device also includes a second abutting member (2′) at the second end of the elongated body (1), which is flexible. The device further includes at least one electrode (16) and a power supply (11) for supplying current to the electrode (16).

### FIELD OF THE INVENTION

The present invention relates to a surgical device to be implanted. In particular, the present invention relates to a gastrointestinal implant that enables to reduce food uptake, and thereby controlling overweight, and accordingly which will be placed in the gastrointestinal tract.

### STATE OF THE ART

Document U.S.-2006/0265021-A1 describes a gastrointestinal implant in the form of a rigid capsule comprising a cavity wherein the stomach wall is sucked. The sucked tissue is penetrated with a pin holding the capsule in place. The pin forms the electrode. This manner of attaching such type of device does not allow a long-term anchoring. It is moreover used for pHmetric capsules such as the one disclosed in U.S. Pat. No. 6,689,056-B1, capsules which are foreseen to be naturally detached after several days.

Document U.S. Pat. No. 7,020,531-B1 describes a capsule similar to the one disclosed in US-2006/0265021-A1 but wherein the pin is flexible. This type of capsule, due to its shape, cannot be implanted in sphincters such as the pyloric sphincter. The device includes a chamber for receiving tissue of the stomach wall for attachment where a vacuum pressure is applied through the chamber to draw the tissue into the chamber.

Document WO-02089655-A2 describes a stimulation device having surface electrodes. This device is submucosally implanted in a pocket created for that purpose between the mucosa and the muscularis (creation of the pocket by liquid injection, dissection, insertion and fixation of the implant, suture). The device includes means for maintaining the device in proper orientation.

### AIMS OF THE PRESENT INVENTION

The present invention aims to reduce food uptake in particular in case of obesity (more particularly, in case of overweight).

The present invention aims to provide a device which can be implanted in the gastrointestinal tract and in particular in sphincters such as the pyloric sphincter.

The purpose of the present invention is to provide a device and a method of implanting such device which can be used for long term periods in particular periods up to several months at least six months preferably up to several years typically five years.

The present invention is particularly to provide a device and a method of implanting such device which can be applied to the pyloric sphincter which is submitted to contraction and relaxation steps.

### SUMMARY OF THE INVENTION

According to one aspect of the present invention, it is provided a gastrointestinal device to be implanted into an animal or human tissue, comprising at least:
an elongated body having a first end and a second end;
at least a first (tissue) abutting member at the first end of the elongated body; characterized in that it further comprises a second (tissue) abutting member at the second end of the elongated body.

Preferably, in a device according to the invention, the elongated body is flexible.

More preferably, the (tissue) abutting members at each end of the elongated body are able to rotate, and/or move longitudinally and/or laterally with respect to each other.

More preferably, said first (tissue) abutting member is fixed at the first end of the elongated body, allowing the second (tissue) abutting member to rotate, and/or move longitudinally and/or laterally with respect to said first (tissue) abutting member.

Preferably, in a device according to the invention, at least one (tissue) abutting member is detachable from, removable from or retractable from the elongated body.

Preferably, a device according to the invention further comprises at least one electrode and a power supply for supplying current to the electrode.

Preferably, a device according to the invention further comprises an electronic circuit for controlling the current delivered by the electrode.

More preferably, the electronic circuit is comprised in one (tissue) abutting member.

Preferably, in a device according to the invention, the power supply is comprised in one (tissue) abutting member.

More preferably, the same (tissue) abutting member comprises the electronic circuit and the power supply.

Preferably, in a device according to the invention, at least one (tissue) abutting member connected to one and/or the elongated body comprises a deploying part having a deployed and a retracted configuration.

Preferably, a device according to the invention further comprises at one end of the elongated body perforating means.

Preferably, the thickness of the (tissue) abutting member is higher than (about) 0.2 mm, and more preferably comprised between (about) 2 mm and (about) 7 mm.

Preferably, the thickness of the (tissue) abutting member is (about) 4 mm.

Preferably, the diameter of the (tissue) abutting member is higher than (about) 2 mm, and more preferably comprised between (about) 4 mm and (about) 15 mm.

Preferably, the diameter of the (tissue) abutting member is (about) 7 mm.

Preferably, the diameter of the elongated body is less than (about) 2 mm, and more preferably comprised between (about) 0.01 mm and (about) 1 mm.

Preferably, the diameter of the elongated body is (about) 0.5 mm.

Preferably, the volume of the (tissue) abutting member is higher than (about) 100 mm³, and more preferably comprised between (about) 25 mm³ and (about) 1000 mm³.

Preferably, the volume of the (tissue) abutting member is (about) 150 mm³.

According to another aspect of the present invention, it is provided a gastrointestinal device to be implanted into an animal or human tissue, comprising at least:
an elongated body having a first end and a second end;
at least a first (tissue) abutting member at the first end of the elongated body; characterized in that:
it further comprises a second (tissue) abutting member at the second end of the elongated body;
the elongated body is flexible;
it further comprises at least one electrode and a power supply for supplying current to the electrode, and thereby providing a stimulator (device).

The present invention provides a device which can be implanted in the gastrointestinal tract and in particular in sphincters such as the pyloric sphincter.

More particularly, the implanted device according to the present invention traverses (or crosses, or perforates) the gastrointestinal tract and in particular muscular layers of sphincters such as the pyloric sphincter.

Preferably, the elongated body of the implanted device is completely inserted in said sphincters.

Preferably, the elongated body of the implanted device is able to perforate (or perforating) said sphincters such that part of said elongated body is coming out of said sphincters.

In yet another aspect of the present invention, it is provided a gastrointestinal device (or an implant) for controlling overweight, to be implanted into an animal or human tissue, comprising at least:
an elongated body having a first end and a second end;
at least a first (tissue) abutting member at the first end of the elongated body; characterized in that it further comprises a second (tissue) abutting member at the second end of the elongated body.

According to still another aspect of the present invention, it is provided a gastrointestinal device for controlling overweight, to be implanted into an animal or human tissue, comprising at least:
an elongated body having a first end and a second end;
at least a first (tissue) abutting member at the first end of the elongated body; characterized in that:
it further comprises a second (tissue) abutting member at the second end of the elongated body;
the elongated body is flexible;
it further comprises at least one electrode and a power supply for supplying current to the electrode.

According to another aspect, the present invention relates to a method for implanting a device in the gastrointestinal tract and in particular in a sphincter such as the pyloric sphincter, comprising the steps of:
providing a device to be implanted into an animal or human tissue, comprising at least an elongated body having a first end and a second end; at least a first (tissue) abutting member at the first end of the elongated body; characterized in that it further comprises a second (tissue) abutting member at the second end of the elongated body;
introducing said device into the gastrointestinal tract in order to place it close to the tissue in which the device will be anchored;
perforating the tissue in which the device will be anchored;
connecting or attaching said device to the tissue.

Preferably, a method according to the invention further comprises a step of:
stimulating with current pulses said tissue.

Alternatively, in a method according to the invention, no electrical stimulation is performed.

Preferably, a method according to the invention further comprises a step of
detaching one (tissue) abutting member from the elongated body in order to have a free (tissue) abutting member and an elongated body having its perforating means present at the free end.

More preferably, the step of detaching is performed before the step of introducing said device.

More preferably, the step of detaching or removing is performed after the step of introducing said device.

Preferably, in a method according to the invention, the step of introducing said device is performed with a deployable part of the device whereby the deployable part of the device is in its retracted configuration.

More preferably, the step of attaching the device to the tissue is performed by deploying the deployable part of the device.

In still another aspect of the present invention, it is provided a gastrointestinal device (or an implant), made (or obtainable) according to the method as above described.

In yet another aspect of the present invention, it is provided a gastrointestinal device (or an implant) for controlling overweight, used (or obtainable) according to the method as above described.

According to another aspect, the present invention relates to the use of a method as above described for the manufacture of a gastrointestinal device (or an implant) to be implanted into an animal or human tissue.

According to yet another aspect, the present invention relates to the use of a method as above described for the manufacture of a gastrointestinal device (or an implant) for controlling overweight, to be implanted into an animal or human tissue.

### BRIEF DESCRIPTION OF THE DRAWINGS

The following drawings represent different aspects of the present invention. The drawings are described in specific embodiments. However, the present invention can also relate to a combination of several features or elements described in several different embodiments and which can be associated or combined in new extra embodiments.

FIG. 1a shows a general view of an implant according to the present invention anchored in the pyloric sphincter.

FIG. 1b shows a general view of an implant according to the present invention anchored in the pyloric sphincter, further depicting perforating means at one end of the elongated body, and depicting an electrode comprised in one of the (tissue) abutting members, said electrode and said one of the (tissue) abutting members being in direct contact with the human or animal tissue.

FIG. 2a shows a view in a longitudinal section of a gastrointestinal implant according to a preferred embodiment which comprises an elongated body connected to a (tissue) abutting member comprising an electronic circuit. A second (tissue) abutting member comprising a power supply can be connected to the free end of the elongated body.

FIG. 2b shows a view in a cross section of the second (tissue) abutting member comprising a power supply.

FIG. 3 shows a view in a longitudinal section of the elongated body according to a preferred embodiment.

FIG. 4a shows a detailed view of one end of the elongated body having an external thread and corresponding to a (tissue) abutting member that comprises a hollow having an internal thread into which the end of the elongated body is screwed.

FIG. 4b shows a detailed view of one end of the elongated body having a grove and a corresponding (tissue) abutting member that comprises a hollow having a shoulder onto which the end of the elongated body is clipped.

FIG. 5 shows a view in a longitudinal section of an implant according to another embodiment of the present invention.

FIGS. 6a-c show a view in a cross section of several examples of (tissue) abutting member.

FIG. 7 shows the elongated body connected at one end to a plug shape (tissue) abutting member and at the other end to a (tissue) abutting member comprising a deploying part, the deploying part being in its retracted configuration.

FIG. 8 shows the implant anchored in a tissue wall, the implant comprising at one end a plug shape (tissue) abutting member and at the other end a (tissue) abutting member comprising a deploying part, the deploying part being in its deployed configuration.

FIGS. 9a-c show several examples of an implant with possible deploying part.

FIG. 10 shows a view in longitudinal section of a set of two implants anchored in a tissue wall, the implants being electrically connected.

FIGS. 11a and 11b show two views in a frontal section of the implant anchored into the pyloric sphincter, the views being respectively taken from the stomach and from the duodenum.

FIG. 12a shows a view of a longitudinal section of an implant—anchored into a tissue wall—comprising two elongated bodies that are connected to the same (tissue) abutting members.

FIG. 12b shows a view of the frontal section of an implant—anchored into the pyloric sphincter—comprising two elongated bodies that are connected to the same (tissue) abutting members.

FIGS. 13a and 13b display two examples of a current pulse train that can be delivered by the implant to a tissue.

FIG. 14 shows a lateral view and an apical view of a (tissue) abutting member comprising an external case made of a grid.

FIGS. 15a-d show several steps of the implantation procedure of a device according to the present invention in the pyloric sphincter said procedure comprising 4 major steps.

### DETAILED DESCRIPTION OF THE PRESENT INVENTION

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

The present invention relates to an implant for controlling food uptake, in particular for animals or human beings displaying morbid obesity (or overweight).

More particularly, the present invention relates to an implant for controlling overweight.

In the context of the present invention, “obesity” (or a person being obese) refers to (a person having) “overweight”, i.e. a person having a Body Mass Index (or BMI) higher than 25.

The (implant according to the) present invention can be placed inside a human or animal body using standard surgical techniques such as laparoscopy or standard endoscopy techniques such as endoluminal digestive endoscopy.

The implant according to the present invention can be placed inside the gastrointestinal tract, in particular inside the abdominal cavity, in particular trough tissue walls delimitating the gastrointestinal tract or the abdominal cavity.

Preferably, the implant can be anchored in any sphincter of the gastrointestinal tract such as the pyloric sphincter, the lower esophageal sphincter or the internal anal sphincter.

More preferably, the implant is anchored in the pyloric sphincter.

In comparison to known techniques for electrical stimulation of the gastrointestinal tract, various embodiments of the invention may provide one or more advantages. For example, a device (or implant) in accordance with the invention can be deployed within the patient without the need for surgical procedures. Rather, the device can be endoscopically placed at a location within the gastrointestinal track via the patient's nose or mouth or anus.

Therefore, in addition to avoiding surgery, there is no need for leads to extend from the patient's nose or mouth or anus. On the contrary, the entire device is contained within the gastrointestinal tract and includes a fixation structure to attach the device directly to tissue within the gastrointestinal tract. Consequently, a device in accordance with the invention eliminates the need for surgery and reduces patient discomfort. In addition, the device may be readily implanted for long term treatment, offering a more convenient therapy for patients suffering from symptoms such as nausea or vomiting following surgery or chemotherapy.

The device also may be suitable for trial (electrical) stimulation to predict the efficacy of chronic implantation of a gastrointestinal (electrical) stimulation device for a given patient.

As a further advantage, the (electrical) stimulation device may even be used as a preventative treatment for nausea or vomiting, thereby reducing in-house medical expenses associated with treatment of such symptoms.

In some embodiments, the device may be endoscopically detachable or possibly endoscopically retrievable, requiring no surgical procedure for explant.

The implant is arranged to be reliably anchored in the tissue(s) so that it can stay in place for a long period of up to several months (at least 6 months) and preferably of up to several years, typically five years.

The implant is designed so as to be used for perforating the targeted tissue(s) during implant insertion.

The implant comprises at least one (tissue) abutting member that is fixed at one end of an elongated body.

Preferably, the implant comprises at each end a (tissue) abutting member for avoiding the detachment of the implant from the tissue where it is anchored.

In the context of the present invention, the term ‘(tissue) abutting member’ refers to any mechanical means that is arranged in a way, that when the implant is anchored in a tissue, the implant cannot be removed from the tissue without a voluntary human intervention.

Preferably, the two abutting members are two separated members not in direct contact.

In particular, it (or the implant) cannot be removed from the tissue without either removing at least one of said (tissue) abutting members or in the case the abutting member comprises a deploying part, without putting the deploying part back in its retracted configuration.

Preferably, the elongated body (of the implant) is flexible (or elastic, or pliable).

Preferably, the elongated body (of the implant) is a spiral, or a coaxial device.

Preferably, the (tissue) abutting members are attached to (but are not fixed) at each end of the elongated body, such that said (tissue) abutting members are able to (at least) rotate with respect to each other, and/or move longitudinally and/or laterally with respect to each other (or allowing said (tissue) abutting members to (at least) rotate with respect to each other, and/or move longitudinally and/or laterally with respect to each other).

More particularly, the elongated body (of the implant) is flexible (or elastic, or pliable), allowing the (tissue) abutting members at each end of the elongated body to (at least) rotate with respect to each other, and/or to move longitudinally and/or laterally with respect to each other.

Preferably, one (or a first) (tissue) abutting member is fixed at one (or the first) end of the elongated body, while the other (or the second) (tissue) abutting member is attached to (but is not fixed) at the other (or the second) end of the elongated body, allowing the second (tissue) abutting member to (at least) rotate with respect to the first (or fixed) (tissue) abutting member, and/or move longitudinally and/or laterally with respect to said first (or fixed) (tissue) abutting member.

More particularly, the elongated body (of the implant) is flexible (or elastic, or pliable), allowing the second (tissue) abutting member to (at least) rotate with respect to the first (or fixed) (tissue) abutting member, and/or move longitudinally and/or laterally with respect to said first (or fixed) (tissue) abutting member.

Preferably, the term ‘flexible’ means that a 12 mm long elongated body supported on each end would deform by at least 1 mm when subjected to a 20 mN normal force applied to the middle.

More preferably, the term ‘flexible’ (or elastic, or pliable) means that the elongated body is able to follow tissue movements—such as contraction and relaxation steps—without inducing tissue damages.

This is particularly important in case of an implantation (of the implant) in the pyloric sphincter which is a tissue involving a lot of movements and/or stresses, especially for long term implantation.

Preferably, the length of the elongated body (of the implant) is predetermined depending on the physiological and anatomical parameters of the person who wears the implant but the length of the elongated body between two (tissue) abutting members can be modified during the anchoring procedure or after the anchoring procedure.

More preferably, the length between the two (tissue) abutting members of the elongated body can dynamically vary with the thickness of the tissue in which it is anchored.

For example in the case of an implant anchored in the pyloric sphincter, the length between the two (tissue) abutting members of the elongated body can change according to the contraction-relaxation cycle of the sphincter.

Preferably, the length between the two (tissue) abutting members of the elongated body can be modified in order to have at least one (tissue) abutting member adhering to (or in contact with) the surface(s) of the tissue in which the implant is anchored.

Preferably, at least one end of the elongated body comprises means (40, or 40′) for perforating the tissue where the implant is to be anchored.

According to a preferred embodiment, the implant can be a stimulator device (for electrical stimulation of the gastrointestinal tract).

Preferably, the implant comprises an elongated body—having two ends—comprising at least one electrode for delivering current or voltage to the tissue.

Preferably said elongated body is arranged so as to comprise at least one and preferably two electrodes that put the electrical conducting material of the electrodes into contact with the tissue(s) (or with the sphincters) in which the implant is anchored.

In the present invention, the term “electrode” refers to an electrical conducting material used to make contact with a non-metallic part of a (electrical) circuit.

Therefore, in order to enable electrical contact, at least one abutting member should be in direct contact with the human or animal tissue and is accordingly defined as a tissue abutting member.

More preferably, the elongated body comprises at least one pair of bipolar electrodes.

Alternatively, at least one electrode is located on the (tissue) abutting member(s) at a place where it can be in contact with the tissue to (electrically) stimulate.

The electrode(s) is/are preferably made of a corrosion resistant metal or alloy such as platinum, iridium, gold, tantalum, titanium, stainless steel or an alloy of one or more of these metals, e.g. a platinum/iridium alloy.

Preferably, the electrical conductivity of the electrode(s) belongs to the interval between 50 000 Ohm⁻¹cm⁻¹ and 500 000 Ohm⁻¹cm⁻¹ at room temperature (20° C.)

Preferably, the electrode(s) is/are able to allow a charge injection whose value belongs to the interval between 0.04 mC/cm² and 20 mC/cm² without undergoing electrode degradation—such as corrosion—during current flow.

Preferably, the energy for producing the current is supplied by a power supply that is fixed to one end of the elongated body or preferably comprised either in one of (tissue) abutting member(s), or in the elongated body, or even in both.

The power supply can be, for example, a battery, a rechargeable battery, a temporary energy storage such as a capacitor or a buffer storing energy radiated from an external source.

Advantageously, the current delivery is controlled by an electronic circuit that is fixed at one end of the elongated body and preferably is comprised in one (tissue) abutting member.

Advantageously, the electronic circuit is controlled by an external device which is located outside the human or animal body.

More advantageously, the external device does not have any physical connection with the electronic circuit and communicate with the electronic circuit using wireless means such as Wifi, RFID, . . .

Preferably, the electronic circuit and the power supply are fixed at the same end of the elongated body.

Alternatively, the electronic circuit is fixed at one end of the elongated body, whereas the power supply is fixed at the other end of the elongated body.

The implant is arranged to deliver an electrical current—such as current pulses—preferably via a pulse generator to the tissue(s) in which it is implanted.

The pulse generator and electrodes can be mounted within a common device housing, such as a capsule.

Preferably, the electrical current is generated by a pulse generator which forms current pulse trains that are for example short pulse trains or long pulse trains.

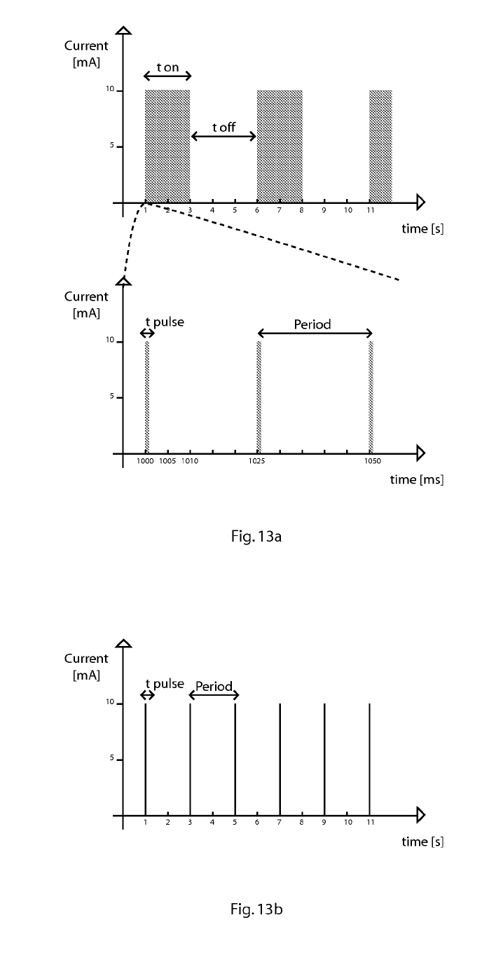

These two examples of pulse trains are represented in FIGS. 13a and 13b, respectively.

The short pulse trains lasting for a time tₒₙ can be made of sequences of individual pulses and interrupted by (a) resting period(s) lasting for a time t_{off} (see FIG. 13a).

Each individual pulse lasts for a period tₚᵤₗₛₑ.

Preferably, the value of tₚᵤₗₛₑ belongs to the interval comprised between 20 microseconds and 2 milliseconds.

The period ‘Period’ between two consecutive pulses belongs to the interval comprised between 10 milliseconds and 100 milliseconds.

The long pulse trains can be made of individual current pulses lasting for a period tₚᵤₗₛₑ occurring with a period called ‘Period’ (see FIG. 13b).

Preferably, the tₚᵤₗₛₑ value belongs to the interval comprised between 2 milliseconds and 500 milliseconds.

Preferably, the ‘Period’ value belongs to the interval comprised between 1 second and 30 seconds.

Preferably, the maximum value of the current delivered to the tissue is not exceeding a predetermined value corresponding to the maximal value of current that does not induce iatrogenic effects to the person wearing the implant, said effect being characterized for example by pain or discomfort such as nausea, cramping or bloating.

For example, this value is currently considered as being close to or equal to 20 mA [see “What are the Yanks doing?” The U.S. experience with implantable gastric stimulation (IGS) for the treatment of obesity—Update on the ongoing clinical trials; Scott A. Shikora; Obesity Surgery, 14, s40-48; 2004] but may vary between individuals.

More preferably, the value of the maximum current should not exceed 50 mA.

Preferably, each end of the elongated body has connection means for realizing mechanical and/or electrical connections between the elongated body and the (tissue) abutting member(s).

Preferably, the (tissue) abutting members have connection means for realizing mechanical and/or electrical connections between the elongated body(s) and themselves.

Preferably, the elongated body and the (tissue) abutting member(s) are connected in a screw-nut fashion.

Alternatively, the elongated body and the (tissue) abutting member(s) are connected by clipping the elongated body end into the (tissue) abutting member(s).

Preferably, after being anchored in a tissue, the (tissue) abutting members can be removed and replaced throughout the implant life in the tissue by means of human intervention with dedicated tools such as endoscopic tools.

For example, such procedure of removal can be used when replacing a battery which is at a too low power level.

Preferably, at least one (tissue) abutting member fixed to one end of the elongated body comprises a deploying part.

Preferably, said deploying part has at least a retracted configuration and a deployed configuration, each configuration being preferably a 2-dimensional and more preferably a 3-dimensional configuration.

Preferably, the deploying part comprises at least one member that is arranged to deploy, thereby increasing the maximum orthogonal section of the implant.

Alternatively, the deploying part comprises at least two members that are arranged to deploy, thereby increasing the maximum orthogonal section of the implant.

In the present case, the term ‘orthogonal section’ is intended to mean a section at one point defined by a plan perpendicular to the tangent to the longitudinal axis of the elongated body.

Preferably, the orthogonal section to the longitudinal axis of the elongated body is any 2-dimensional geometrical shape defined by one or two closed curves.

Preferably, the orthogonal section of the elongated body is a disc.

Preferably, the orthogonal section of the elongated body is a ring.

Alternatively, the orthogonal section of the elongated body is a rectangle.

Alternatively, the orthogonal section of the elongated body is a square.

Preferably, the orthogonal section of the elongated body is constant along the longitudinal axis of the elongated body.

Alternatively, the orthogonal section of the elongated body varies along the longitudinal axis of the elongated body.

In the retracted position, the deploying part has preferably perforating means (40, or 40′) that enable to perforate the tissue during the anchoring procedure.

Preferably, the elements of the implant are embedded in a surface layer made of biocompatible materials.

Preferably, the elements of the implant are made of biocompatible materials.

More preferably, the elements of the implant are embedded in or made of materials that resist to low pH medium.

More preferably, the elements of the implant are embedded in biocompatible materials that resist to low pH medium.

In the present invention, the term “low pH” is intended to mean pH under 4. Such a low pH is encountered in the stomach where the pH is between 1 and 4.

For example, the elements of the implant are embedded in or made of materials such as titanium (alloys) or stainless steel, biocompatible polymers belonging to the polyolefin family such as high density polyethylene (HDPE), linear low density polyethylene (LLDPE), polypropylene (PP), ultra high molecular weight polyethylene (UHMWPE), biocompatible polymers belonging to the fluoropolymer family such as polytetrafluoroethylene (PTFE), fluorinated ethylene propylene (FEP), or biocompatible silicons or elastomers.

Preferably, the (tissue) abutting members of the implant are embedded or made of Ti6A14V ELI (grade 23).

Preferably, the (tissue) abutting members have a maximum orthogonal section that is strictly superior to the maximum orthogonal section of the elongated body.

Preferably, the orthogonal section of the (tissue) abutting member is at least twice the orthogonal longitudinal axis of the elongated body.

In the present invention, the term proximal end of the elongated body is intended to mean the end that is proximal to the opening through which the implant has been inserted into the human or animal body when implanted in the human or animal body.

For example, when luminal endoscopy is used for performing the placement of the implant, the proximal end of the elongated body is the end proximal relatively to the mouth of the treated human or animal when implanted in the human or animal body.

In the present invention, the term distal end of the elongated body is intended to mean the end that is distal to the opening through which the implant has been inserted into the human or animal body.

### DETAILED DESCRIPTION OF PREFERRED EMBODIMENTS OF THE PRESENT INVENTION

Each example describes a preferred embodiment according to the present invention, each comprising features or elements (described in some details) which can be associated or combined in the other embodiments (not necessarily described but covered by the present invention).

### EXAMPLE 1

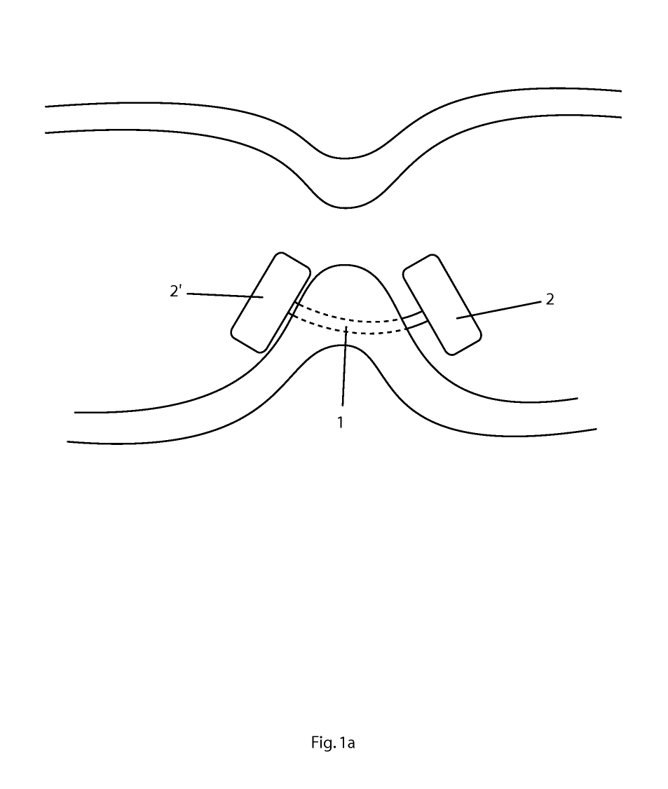

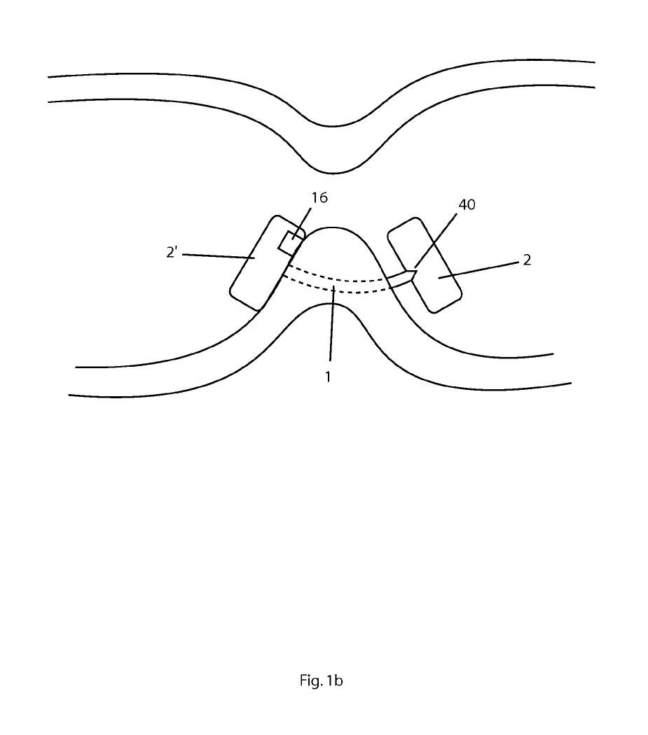

The implant presented in FIG. 1 comprises two tissue abutting members (2 and 2′) having a plug shape and respectively connected to one elongated body (1) end.

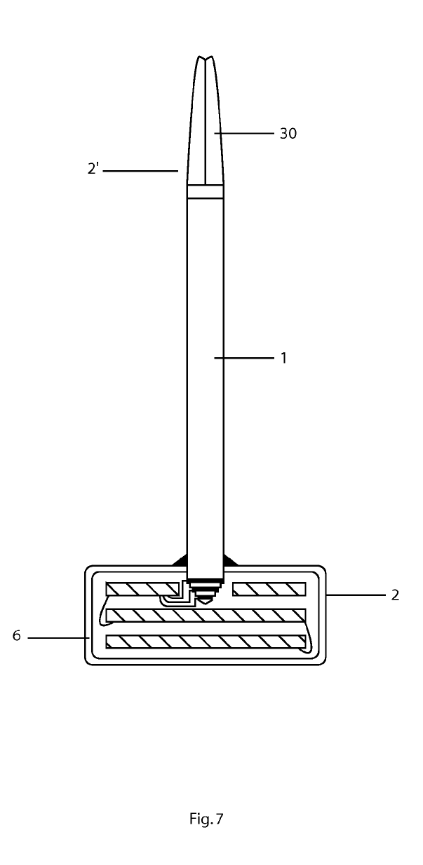

For example, the tissue abutting members (2 and 2′) that are fixed to the ends have a section as the ones displayed in FIG. 7.

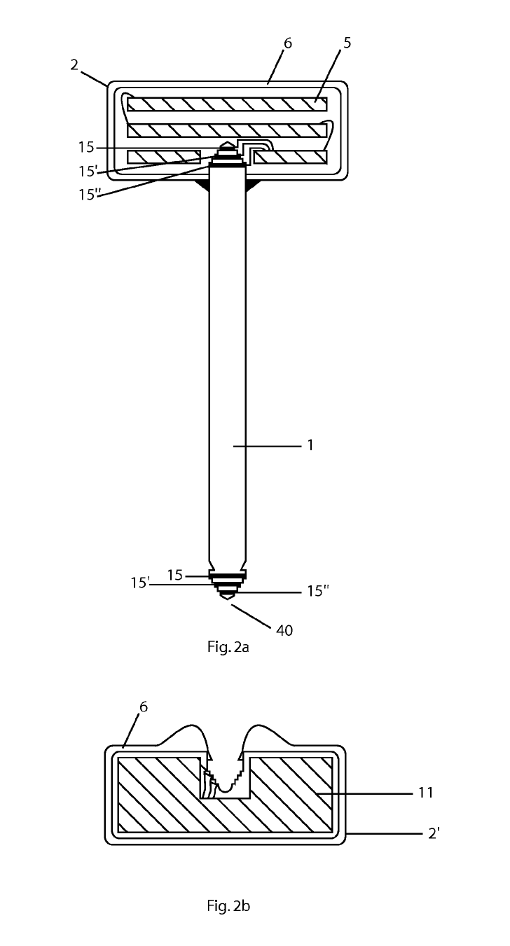

As depicted at FIGS. 2a and 2b, these (tissue) abutting members comprise a circuit (5) and a power supply (11) respectively.

Furthermore, the (tissue) abutting members as depicted in FIGS. 2a and 2b further comprise isolation material (6).

Before being anchored in the tissue(s), at least one (tissue) abutting member is not fixed (or is even not attached) at one end of the elongated body.

The perforating means of the elongated body are sharp elements located at the tip of the elongated body ends that enable to perforate the tissue in which the implant has to be anchored.

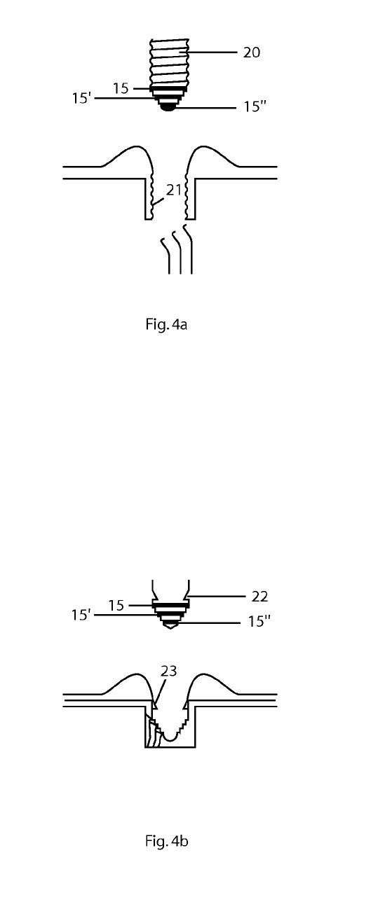

Preferably—as depicted in FIG. 4a—the connection means of the elongated body (1) and the (tissue) abutting member(s) (2) are an outer thread (20) located at least on one end of the elongated body and an inner thread (21) inside a hollow cavity in the (tissue) abutting member (2), respectively.

Alternatively—as depicted in FIG. 4b—the connection means of the elongated body (1) and the (tissue) abutting member(s) (2) are a grove (22) in at least one end of the elongated body (1) and a shoulder (23) inside a hollow cavity in the (tissue) abutting member (2), respectively, the shape of the shoulder (23) being complementary to the shape of the grove (22) in the end of the elongated body.

Preferably—as depicted in FIGS. 4a and 4b—, the (tissue) abutting member(s) that are screwed or clipped to the elongated body comprise contacting means (or electrical contacts) (15,15′,15″) for enabling current to flow through the connection between the (tissue) abutting members (2,2′) and the elongated body (1).

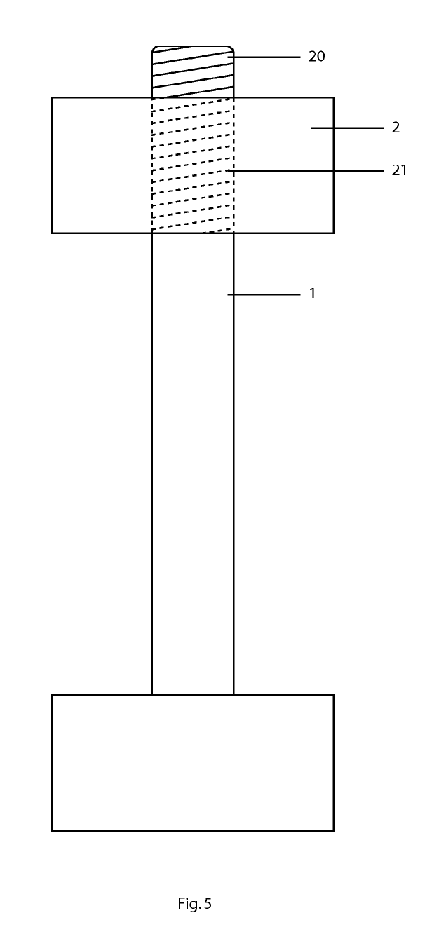

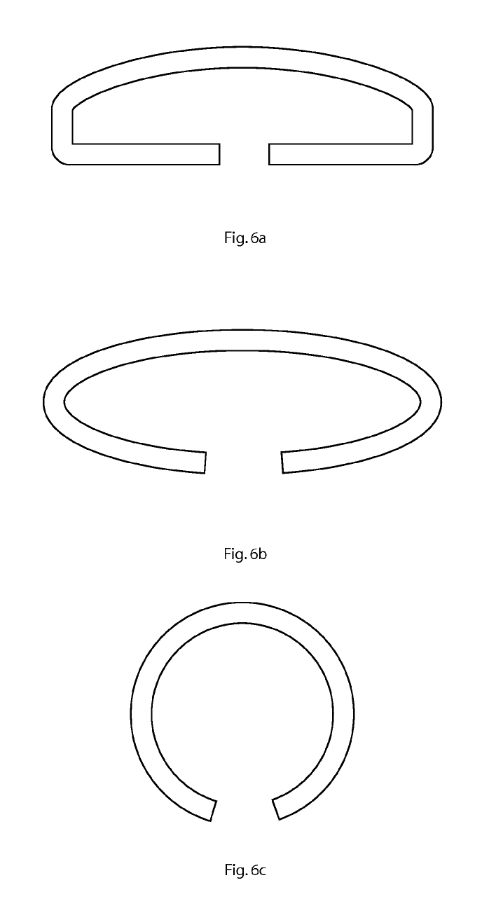

Alternatively—as depicted in FIG. 5—the connection means of the elongated body (1) and the (tissue) abutting member(s) (2) are an outer thread (20) located at least on one end of the elongated body and an inner thread (21) inside a channel having two openings in the (tissue) abutting member (2), respectively.

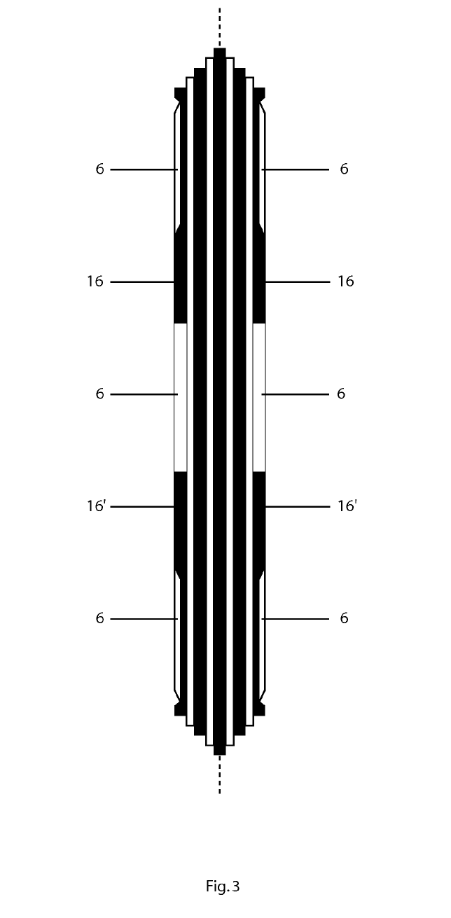

Preferably—as depicted in FIG. 3—, the elongated body is arranged so as to comprise at least two electrodes (16,16′) (and isolation material (6)).

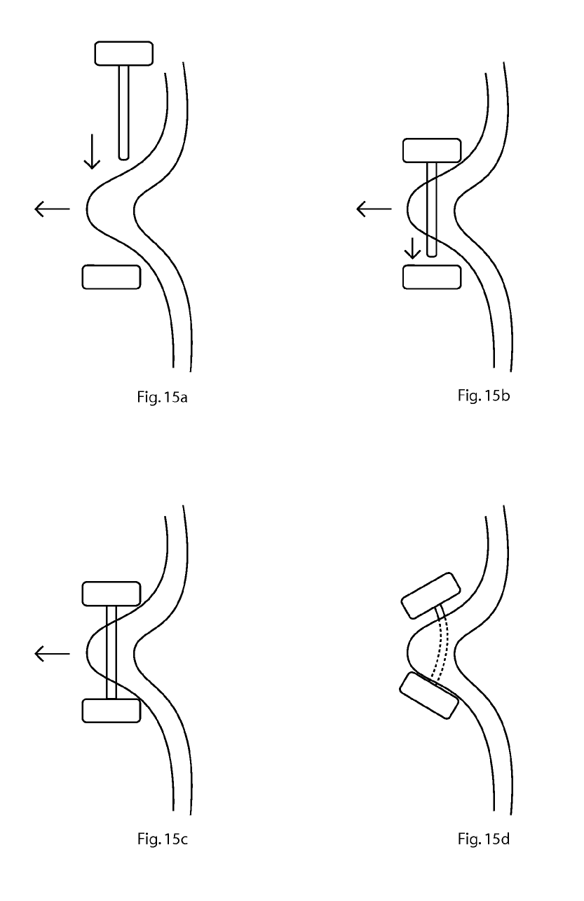

The method for anchoring said implant in the target tissue can comprise the following steps (FIG. 15):
inserting (or implanting) the implant into the body close to the tissue in which it will be anchored such as the pyloric sphincter (FIG. 15a);
perforating the tissue by means of the perforating means of the implant while applying a (lifting) force (arrow) to the tissue (FIG. 15b);
connecting the free tissue abutting member to the elongated body end, while applying a (lifting) force (arrow) (or while maintaining the (lifting) force applied in the previous step) to the tissue (FIG. 15c);
relaxing the force applied to the tissue during the previous steps (FIG. 15d).

### EXAMPLE 2

The implant presented in FIGS. 7, 8, and 9 comprises one plug shape (tissue) abutting member (2) and one (tissue) abutting member (2′) that has a deploying (or deployable) part (30), both elements being connected to separate elongated body ends.

In this case, the (tissue) abutting member comprising the deploying part (30) is connected to the distal end of the elongated body prior to the perforation of the tissue(s) by the implant.

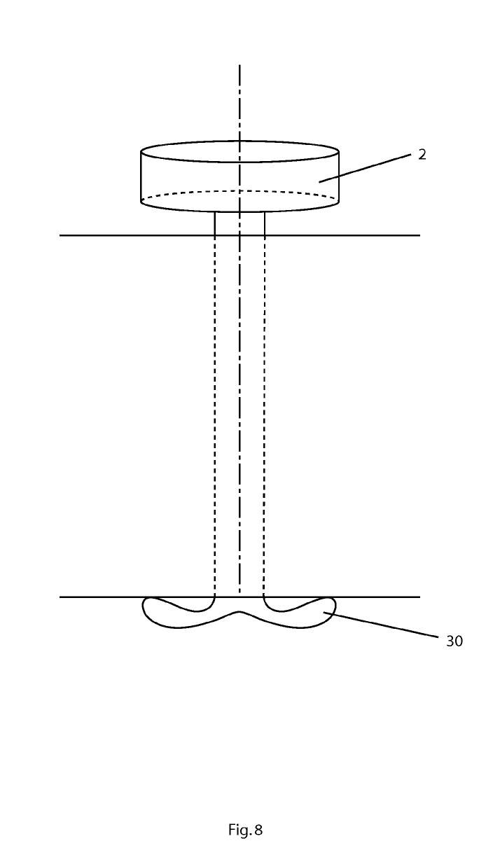

Preferably, said deploying part (30) has at least a retracted configuration—as depicted in FIG. 7—and a deployed configuration—as depicted in FIG. 8—, both configurations being preferably two 3-dimensional configurations.

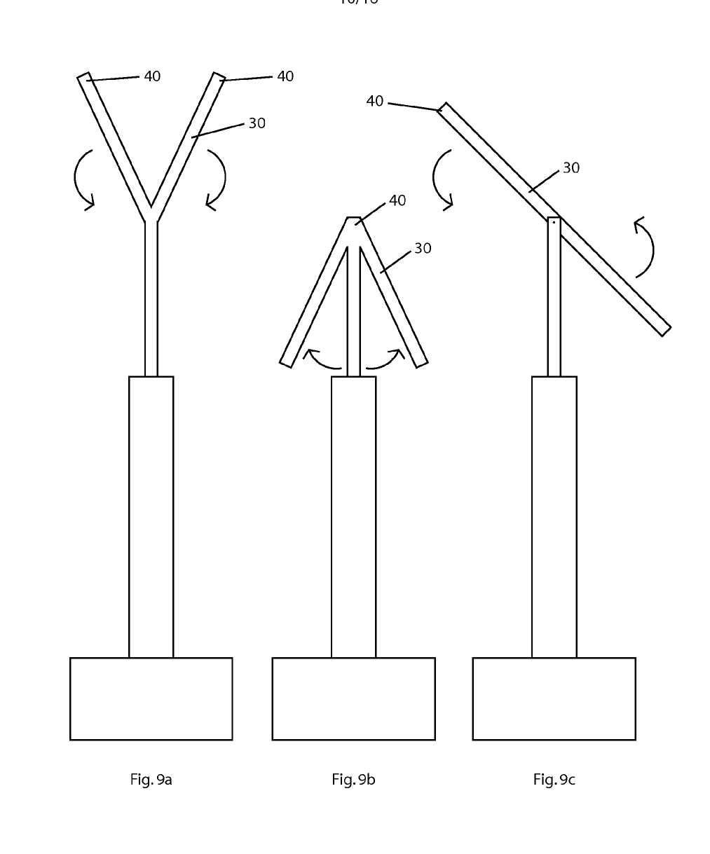

Preferably, the deploying part comprises at least one member that is arranged to deploy, thereby increasing the maximum orthogonal section of the implant, as depicted in FIG. 9c.

Alternatively, the deploying part comprises at least two members that are arranged to deploy, thereby increasing the maximum orthogonal section of the implant, as depicted in FIGS. 9a and 9b.

In the retracted position as shown in FIG. 7, the deploying part has perforating means that enable to perforate the tissue during the anchoring procedure.

A tissue abutting member can be connected to the proximal end of the elongate body either prior to the tissue perforation or after it.

The method for anchoring said implant in the target tissue can comprises the following steps:
bringing the implant close to the tissue in which it will be anchored such as in the stomach if the implant has to be anchored in the pyloric sphincter;
perforating the tissue by means of the perforating means of the deploying part of the implant;
deploying the deploying part of the distal element;
connecting the other (tissue) abutting member to the distal elongated body end if it was not connected prior to the perforation of the tissue.

### EXAMPLE 3

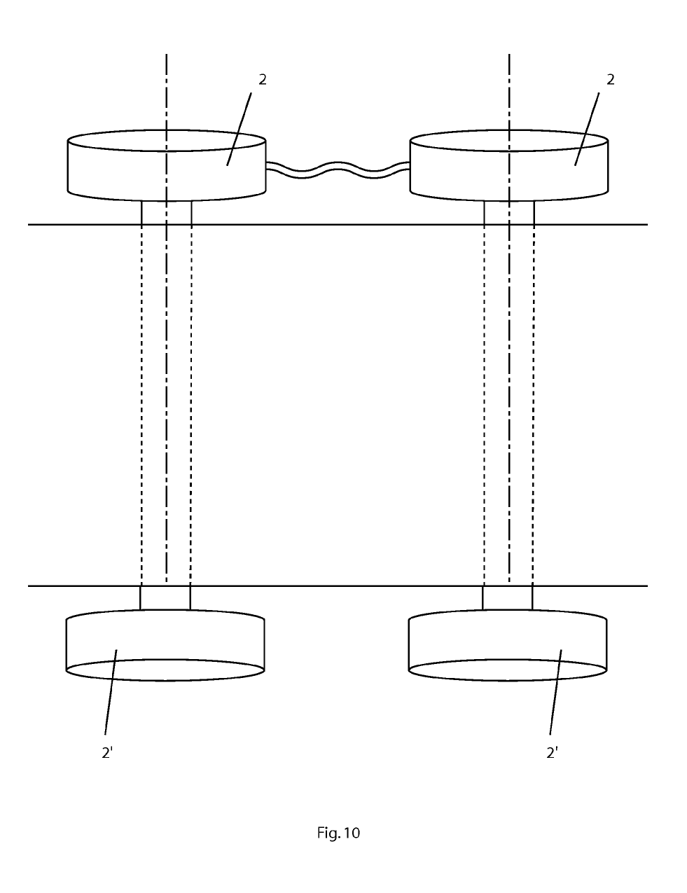

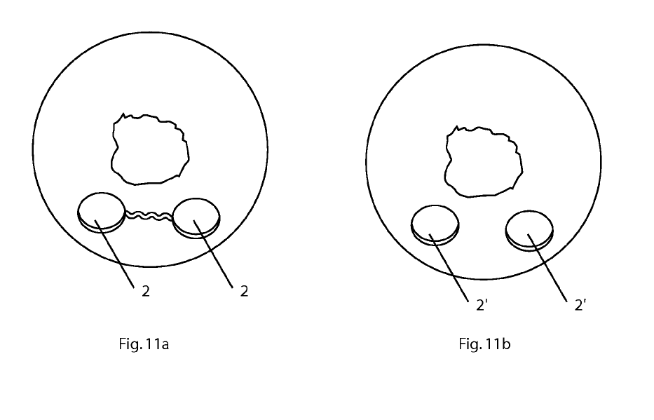

The implant presented in FIGS. 10 and 11 comprises a pair of implants in accordance with the present invention that are connected by a physical connection between two adjacent abutting members respectively belonging to each implant.

### EXAMPLE 4

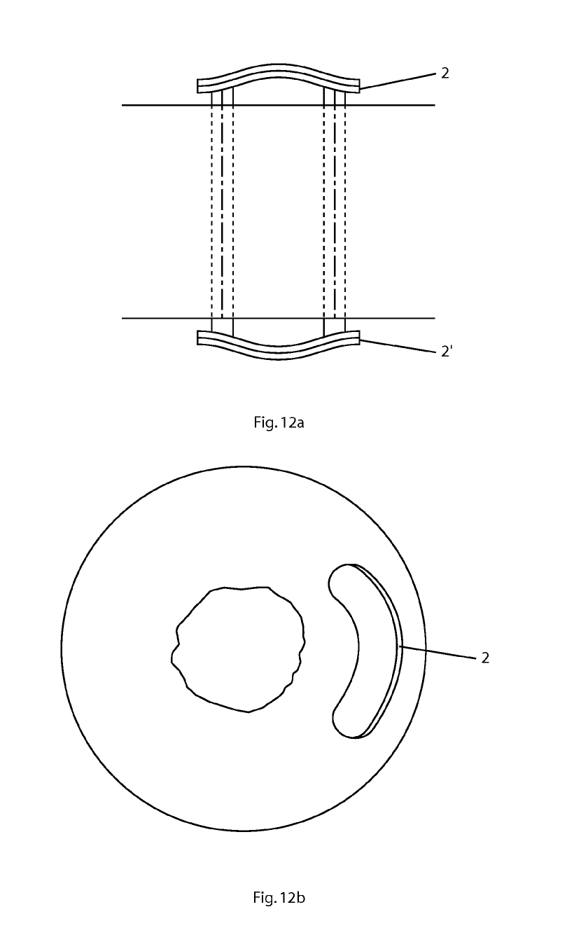

The implant presented in FIG. 12 comprises two elongated bodies that are connected at their respective ends to one common (tissue) abutting member.

### EXAMPLE 5

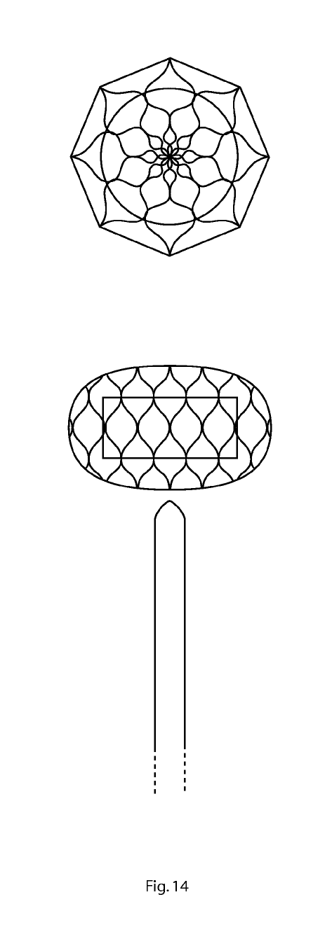

The (tissue) abutting member presented in FIG. 14 comprises an external case made of a grid of biocompatible materials.

The grid is flexible.

The grid has a similar structure to the structure of a stent (or, the structure of the grid is similar to the structure of a stent).

The grid structure is arranged to enable tissue to grow inside it and therefore improving over time the anchoring of the implant in the tissue.

1. A gastrointestinal device to be implanted into an animal or human tissue, comprising:
an elongated body having a first end and a second end;
at least a first abutting member at the first end of the elongated body;
a second abutting member at the second end of the elongated body;
the elongated body is flexible;
at least one electrode and a power supply for supplying current to the electrode.

2. The device according to claim 1, wherein the abutting members at each end of the elongated body are able to rotate, or move longitudinally or laterally with respect to each other.

3. The device according to claim 1, wherein said first abutting member is fixed at the first end of the elongated body, allowing the second abutting member to rotate, or move longitudinally or laterally with respect to said first abutting member.

4. The device according to claim 1, wherein at least one abutting member is detachable from, removable from or retractable from the elongated body.

5. The device according to claim 1, further comprising an electronic circuit for controlling the current delivered by the electrode.

6. The device according to claim 5, wherein the electronic circuit comprises one abutting member.

7. The device according to claim 1, wherein the power supply is comprised in comprises one abutting member.

8. The device according to claim 6, wherein the same abutting member comprises the electronic circuit and the power supply.

9. The device according to claim 1, wherein at least one abutting member connected to one or the elongated body comprises a deploying part having a deployed and a retracted configuration.

10. The device according to claim 1, it further comprising at one end of the elongated body perforating means.

11. A method for implanting a device in a sphincter in the gastrointestinal tract, comprising the steps of:
providing a device according to claim 1;
introducing said device into the gastrointestinal tract to place it close to the tissue in which the device will be anchored;
perforating the tissue in which the device will be anchored;
connecting or attaching said device to the tissue.

12. The method for implanting a device according to claim 11, comprising a further step of:
stimulating with current pulses said tissue.

13. The method for implanting a device according to claim 11, wherein no electrical stimulation is performed.

14. The method for implanting a device according to claim 11, comprising a further a step of
detaching one abutting member from the elongated body to have a free abutting member and an elongated body having perforating means present at the free end.

15. The method for implanting a device according to claim 14, wherein the step of detaching is performed before the step of introducing said device.

16. The method for implanting a device according claim 14, wherein the step of detaching or removing is performed after the step of introducing said device.

17. The method for implanting a device according to claim 1, wherein the step of introducing said device is performed with a deployable part of the device wherein the deployable part of the device is in its a retracted configuration.

18. The method for implanting a device according to claim 17, wherein the step of attaching the device to the tissue is performed by deploying the deployable part of the device.
